(19) United States
(12) Patent Application Publication
(10) Pub. No.: US 2017/0181654 A1
(43) Pub. Date: Jun. 29, 2017

(54) WEARABLE HEART ABNORMALITY SYMPTOM SENSING SMART WATCH, AND SUDDEN HEART ATTACK PREVENTION SYSTEM AND METHOD USING DIGITAL ELECTROCARDIOGRAM (ECG) CORDINATES

(71) Applicant: Moon-Seog JUN, Seoul (KR)

(72) Inventor: Moon-Seog JUN, Seoul (KR)

(21) Appl. No.: 15/391,615

(22) Filed: Dec. 27, 2016

(30) Foreign Application Priority Data
Dec. 28, 2015 (KR) 10-2015-0187860

(51) Int. Cl. A61B 5/04; A61B 5/044; A61B 5/0408; A61B 5/00; A61B 5/0452

(52) U.S. Cl. CPC A61B 5/04012; A61B 5/681; A61B 5/0452; A61B 5/04085; A61B 5/044

(57) ABSTRACT

A sudden heart attack prevention method using an electrocardiogram (ECG) is provided. The method includes obtaining a digital ECG electrical signal, generating digital ECG (X, Y) pattern information by processing the digital ECG electrical signal, sensing whether the digital ECG (X, Y) pattern information deviates from a normal condition, when the digital ECG (X, Y) pattern information deviates from the normal condition, comparing the digital ECG (X, Y) pattern information and an abnormality symptom type, and when it is determined that the digital ECG (X, Y) pattern information is a heart attack presymptom, informing an acquaintance and a hospital of the digital ECG (X, Y) pattern information. According to a configuration of the present invention, when a sudden heart attack occurs, first-aid treatment can be performed without missing the so-called “golden time.”

### CROSS-REFERENCE TO RELATED APPLICATION

This application claims priority to and the benefit of Korean Patent Application No. 2015-0187860, filed on Dec. 28, 2015, the disclosure of which is incorporated herein by reference in its entirety.

### BACKGROUND

1. Field of the Invention

The present invention relates to a wearable heart abnormality symptom sensing smart watch and a system and method including the wearable smart watch for immediately sensing an abnormal pattern using a digital electrocardiogram (ECG) (X, Y) pattern coordinate database (DB) and preventing a sudden heart attack, and more particularly, to a wearable smart watch which immediately senses/warns of a heart abnormality symptom by processing an entire analog ECG waveform as a time axis X-coordinate and a cardiac voltage axis Y-coordinate (for example, ECG PQRST (X, Y) coordinates) in a digital method when storing the entire analog ECG waveform of the wearable smart watch, storing the processed digital coordinates in the wearable smart watch as a DB, and processing and thus storing the processed digital coordinates using a minimum memory capacity and a simple program. Also, in a normal case, a High-Low pattern stored in an ECG DB of the wearable smart watch is sensed, but in an abnormal case, the present invention relates to a sudden heart attack prevention system and method using digital ECG coordinates for determining whether normal (X, Y) coordinates of a heart have deviated from the High-Low pattern by performing ECG PQRST (X, Y) coordinate processing and comparing the High-Low pattern and an abnormality symptom ECG (X, Y) coordinate, further determining whether the High-Low pattern is similar to an ECG Yellow pattern, which is a heart attack presymptom, or an ECG Red pattern, which is a sudden heart attack symptom, when corresponding to the abnormality symptom, providing ECG information to a hospital in the former case and providing the ECG information to an 119 emergency ambulance (a fire station) in the latter case.

2. Discussion of Related Art

Recently, many people are suddenly dying. Surprisingly, more than 50% of patients that had a sudden heart attack thought that they are healthy. This case is often called sudden death. Since sudden death means that a person who normally does not have abnormalities has a heart attack and dies within one hour of any symptom occurring, it is almost impossible to estimate when the symptom will occur.

About 80 to 90% of sudden deaths are caused by heart disease, and about 10 to 20% are caused by an unknown circumstance, for example, death from overwork. About 80% of causes of a sudden heart dysfunction leading to death are arteriosclerosis of coronary arteries, and a myocardial infarction, and a heart attack which are caused by the arteriosclerosis of coronary arteries. In addition, diseases (cardiomyopathy and myocarditis) of the cardiac muscle itself, an arrhythmia, and a heart valve disease are also causes of a sudden heart dysfunction.

A coronary artery which is narrowed by about 30 to 40% by arteriosclerosis does not usually cause any symptoms, but the arteriosclerosis may be the cause of a cardiac infarction which is a representative disease capable of causing the heart attack. A cardiac infarction is caused by bursting of oil deposits that stick to the inside of blood vessels that are narrowed by about 30 to 40% by arteriosclerosis. Components inside the blood vessels exposed by the bursting of oil deposits and components of the blood come into contact with each other and cause a coagulation reaction, as a result, a sudden thrombus interferes with the flow of blood and completely blocks blood circulation of the coronary artery. A cardiac infarction abruptly occurs in people usually has no symptom of heart disease and in which a result of an electrocardiogram (ECG) taken during a medical examination is normal, and results in death.

Meanwhile, a service which provides health management information by transmitting analog waveform biometric information measured by a mobile sensor module to a management center through a mobile terminal and providing the health management information is disclosed in Korean Patent Publication No. 10-2015-0014411. The service can manage diseases by continuously monitoring a condition of a patient by obtaining an analog biometric signal using a biometric signal measurement module and transmitting the obtained analog biometric signal to a health management center using wired or wireless communication technology.

However, a conventional analog waveform transmission method is a method of monitoring only an ECG waveform and has difficulty immediately predicting a heart attack in a wearable terminal, and there are problems in that analog waveforms which are continuously generated are continuously transmitted from the terminal to a server and are stored and it is difficult to store many of the ECG waveforms which are continuously generated in the wearable terminal.

Accordingly, death by a sudden heart attack can be prevented by storing only an ECG PQRST (X, Y) pattern, sensing an abnormal pattern in a currently generated ECG digital pattern, and the problem of the conventional method can be solved by storing the ECG digital pattern in the wearable device in the form of a High-Low pattern of the ECG PQRST (X, Y) pattern and digitizing an analog monitoring problem.

For example, a computer uses a digital method of transmitting a binary number 010101 by digitizing a high voltage as a binary number 1. Accordingly, a heart is recognized as operating by a voltage, and digitization of applying the digital method of the computer to the ECG PQRST pattern is needed. General doctors monitor an entire flow of a waveform using an ECG X-axis waveform, and determine an abnormality symptom of a patient in an empirical method.

An ECG digital method using the PQRST (X, Y) pattern using a Y-axis voltage in an ECG waveform is needed. That's because that the digital (X, Y) pattern has less storage, can be used for predicting various sudden heart attacks, and can be used for diagnosis.

Further, a heart attack presymptom appears several hours to several days before an emergency. When an actual sudden heart attack occurs, it is not easy to rescue a patient within a short time since oxygen is not supplied to the patient's brain. Accordingly, it is necessary to determine a symptom of each step of a heart attack and suitably respond to the heart attack. Further, it is necessary to cope with a heart attack by closely cooperating with acquaintances or rescue institutions in the vicinity of a patient.

### CONVENTIONAL ART

### Patent Document

(Patent Document 1) Korea Patent Publication No. 10-2015-0014411

### SUMMARY OF THE INVENTION

The present invention is directed to providing a sudden heart attack prevention system and method using digital electrocardiogram (ECG) coordinates which may immediately sense a heart abnormality symptom by performing an ECG PQRST (X, Y) coordinate processing on a time axis X-coordinate and a heart voltage axis Y-coordinate in a digital method instead of a method of storing an entire analog ECG waveform, and storing a small amount of digital coordinates in a wearable terminal, and the system may perform first-aid treatment without missing the so-called “golden time” when an emergency such as a heart attack occurs.

Further, the present invention is directed to providing a sudden heart attack prevention system and method using digital ECG coordinates which may prevent a sudden heart attack by cooperating closely with an acquaintance or a related hospital of a user of a wearable device, and which may immediately rescue the user with help of an emergency rescue institution even when a sudden heart attack occurs.

Moreover, the present invention is directed to providing a sudden heart attack prevention system and method using digital ECG coordinates which may determine and cope with a heart abnormality symptom by classifying a heart abnormality symptom of each step using a digital ECG coordinate deviating from a currently sensed pattern in a normal heart ECG pattern database (DB).

According to one aspect of the present invention in order to achieve the purpose described above, there is provided a sudden heart attack prevention system using digital ECG coordinates including: a system for normally learning using repetitive heart biometric voltage information changed according to a heart health condition of a user and predicting and sensing a sudden heart attack, wherein the sudden heart attack prevention system uses a plurality of wearable heart abnormality symptom sensing smart watches which are installed on a wrist of the user and provide heart biometric voltage information of the user, and the wearable heart abnormality symptom sensing smart watch provides biometric voltage (X, Y) pattern information using the heart biometric voltage information, and the biometric voltage (X, Y) pattern information includes ECG PQRST (X, Y) coordinates as a time axis X-coordinate and a heart voltage axis Y-coordinate.

According to another aspect of the present invention in order to achieve the purpose described above, there is provided a sudden heart attack prevention method using ECG coordinates including: obtaining a digital ECG electrical signal; generating digital ECG (X, Y) pattern information by processing the digital ECG electrical signal; sensing whether the digital ECG (X, Y) pattern information deviates from a normal condition; when the digital ECG (X, Y) pattern information deviates from the normal condition, comparing the digital ECG (X, Y) pattern information and an abnormality symptom type; and when the digital ECG (X, Y) pattern information is determined to be a heart attack presymptom, informing an acquaintance and a hospital of the digital ECG (X, Y) pattern information.

According to still another aspect of the present invention in order to achieve the purpose described above, there is provided a wearable heart abnormality symptom sensing smart watch including: a smart band in which an electrocardiogram (ECG) measurement sensor is installed, and which is configured to measure an ECG signal by coming into contact with a wrist of a user; and a smart device fixed on the wrist using the smart band, configured to process and provide the ECG signal by being in electrical contact with the ECG measurement sensor.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a photograph for describing a relationship between an electrocardiogram (ECG) and a heart attack according to a conventional art;

FIG. 2 is a heartbeat generated by an electric shock and an ECG digital (X, Y) pattern waveform displayed as a result thereof according to the present invention;

FIG. 3 is a block diagram illustrating a configuration of a sudden heart attack prevention system using an ECG according to the present invention;

FIG. 4 is a block diagram illustrating a configuration of a wearable heart abnormality symptom sensing smart watch according to the present invention shown in FIG. 3;

FIG. 5 is an ECG waveform illustrating an ECG pattern representing whether to deviate from a normal condition according to the present invention;

FIG. 6 is a Y-axis voltage table illustrating an abnormality symptom representing whether to deviate from a normal condition according to the present invention; and

FIG. 7 is a flowchart illustrating a method of preventing a sudden heart attack using an ECG according to the present invention.

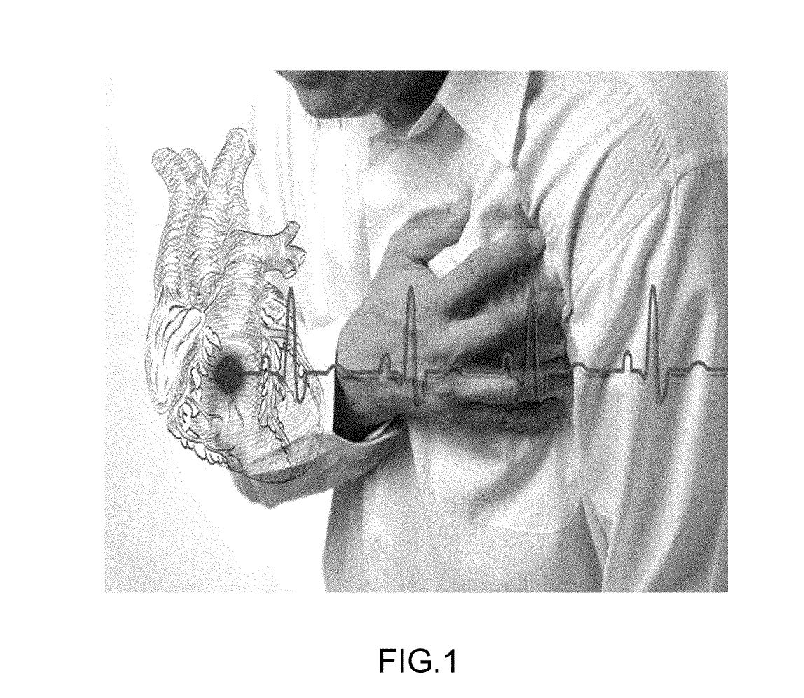

### DETAILED DESCRIPTION OF EXEMPLARY EMBODIMENTS

Advantages and features of the present invention, and a method achieving the advantages and features will be apparent from embodiments of the present invention which will be described hereinafter together with the accompanying drawings. However, the present invention is not restricted by the embodiments which will be described hereinafter and may be implemented in various forms, but the embodiments of the present invention are provided to completely disclose the present invention and are provided to fully illustrate the scope of the present invention to those of ordinary skill in the art, and the scope of the present invention is merely defined by the appended claims. In the accompanying drawings, dimensions and relative sizes of layers and regions may be exaggerated for clarity of description. Throughout the specification, like reference numerals refer to like components.

Hereinafter, a sudden heart attack prevention system using an electrocardiogram (ECG) according to the present invention having a configuration described above will be described in detail with reference to the accompanying drawings.

A sudden heart attack is caused by an electrical problem of a heart. Accordingly, a heartbeat abruptly quickens, and a convulsion occurs instead of blood and oxygen being provided to the brain by the heart pumping.

Accordingly, the present invention may determine a health condition of a heart using an ECG among biometric signals.

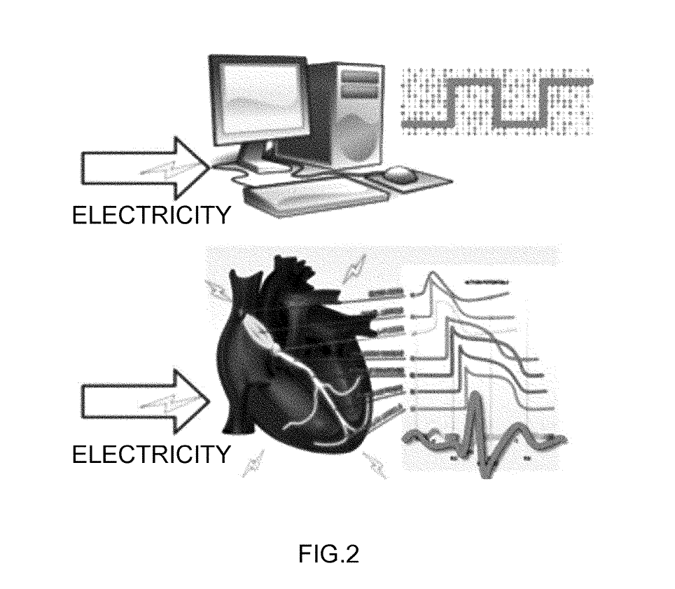

FIG. 2 illustrates a heartbeat generated by an electric shock and an ECG waveform displayed as a result of the electric shock.

An ECG may refer to a recording of an electrical signal generated in a heart. A part called a sinoauricular node may be located in the heart, and the sinoauricular node may be a specific part of the heart controlling a heartbeat by periodically generating electricity and inducing a contraction of the heart.

An electrical signal generated by the sinoauricular node may be transmitted to the entire heart along an electrical conduction system in the heart. A cell consisting of a heart muscle may be contracted by the electrical signal transmitted to each part of the heart, and thus the heart may beat. In this case, a recording of the electrical signal transmitted to the heart by an electrode attached to skin may be called an ECG. The electrode may be attached to various parts of a body, and an electrical phenomenon of each part of the heart may be well recognized.

A system and method for predicting a heart attack presymptom or determining a heart attack symptom and immediately preventing and treating the heart attack using an ECG will be provided.

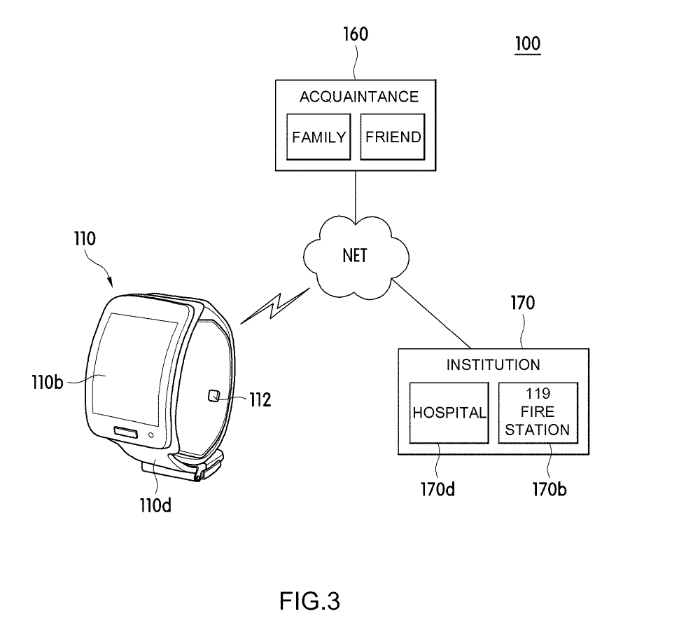

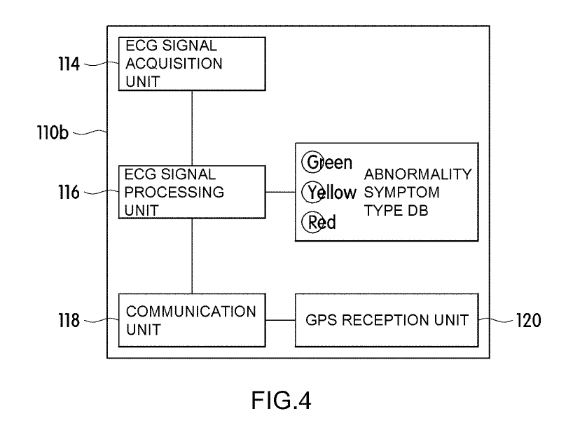

FIG. 3 is a block diagram illustrating a configuration of a sudden heart attack prevention system using an ECG FIG. 4 is a block diagram illustrating a configuration of a smart watch shown in FIG. 3.

Referring to FIGS. 3 and 4, a sudden heart attack preventing system 100 using an ECG according to the present invention may include a plurality of wearable heart abnormality symptom sensing smart watches 110 which generate ECG biometric information.

The wearable heart abnormality symptom sensing smart watch 110 may be provided in the form of a smart watch capable of being worn on a wrist.

The wearable heart abnormality symptom sensing smart watch 110 may include a smart band 110a in which an ECG measurement sensor 112 is installed, and which comes into contact with the wrist of a user and measures an ECG signal, and a smart device 110b which is fixed to the wrist by the smart band 110a, is electrically connected to the ECG measurement sensor 112, and processes the ECG signal.

The ECG measurement sensor 112 may be installed in the form of a biometric electrode inside the smart band 110a which comes into direct contact with wrist skin.

The smart band 110a may be a closed ring type which is flexible and shrinkable. The smart band 110a may be an open ring type which is hard and resilient. Optionally, the smart band 110a may be a fastening type which may be opened or closed.

Accordingly, the smart band 110a may most effectively detect an effect of depolarization and repolarization of a heart, and may be provided in the form of a patch so as to come into close contact with the skin. The smart band 110a may surround the wrist of a user, and the biometric electrode may be installed to face the wrist.

The smart device 110b may be a smart phone, a personal digital assistant (PDA), a handheld personal computer (PC), a hand phone, a home server PC, etc.

The wearable heart abnormality symptom sensing smart watch 110 may include the ECG measurement sensor 112, an ECG signal acquisition unit 114, an ECG signal processing unit 116, and a communication unit 118.

Among the components of the wearable heart abnormality symptom sensing smart device 110, the ECG measurement sensor 112 may be installed in the smart band 110a. The ECG signal acquisition unit 114, the ECG signal processing unit 116, and the communication unit 118, which collect, process, and communicate an ECG signal, may be installed in the smart device 110b, and the ECG measurement sensor 112 and the ECG signal acquisition unit 114 may perform wired communication with each other through the smart band 110a.

The ECG measurement sensor 112 may be configured as one or more biometric electrodes, and the one or more biometric electrode may be configured as a snap electrode which may be used for a long time without stimulating skin of a user.

The ECG signal acquisition unit 114 may be connected to the ECG measurement sensor 112 by a wire and collect and process a digital ECG signal from the biometric electrode. The ECG signal collected by the ECG signal acquisition unit 114 may be a digital electrical signal. As shown in FIG. 2, an electrical signal having a specific voltage waveform may be provided from the biometric electrode.

The ECG signal processing unit 116 may process a waveform having an amplitude (a voltage level) (a Y axis) which continuously changes on a continuous time axis (an X axis) as a digital ECG (X, Y) coordinate pattern, and may classify the processed digital ECG (X, Y) coordinate pattern into various ECG patterns.

An ECG Green pattern type representing a normal condition, an ECG Yellow pattern type representing a sudden heart attack presymptom, and an ECG Red pattern type representing generation of a sudden heart attack may be separately stored in an abnormality symptom type database (DB) by forming the DB, and the ECG signal processing unit 116 may compare the processed ECG pattern and comparable pattern types stored in the abnormality symptom type DB and determine whether the processed ECG pattern has deviated from the normal condition.

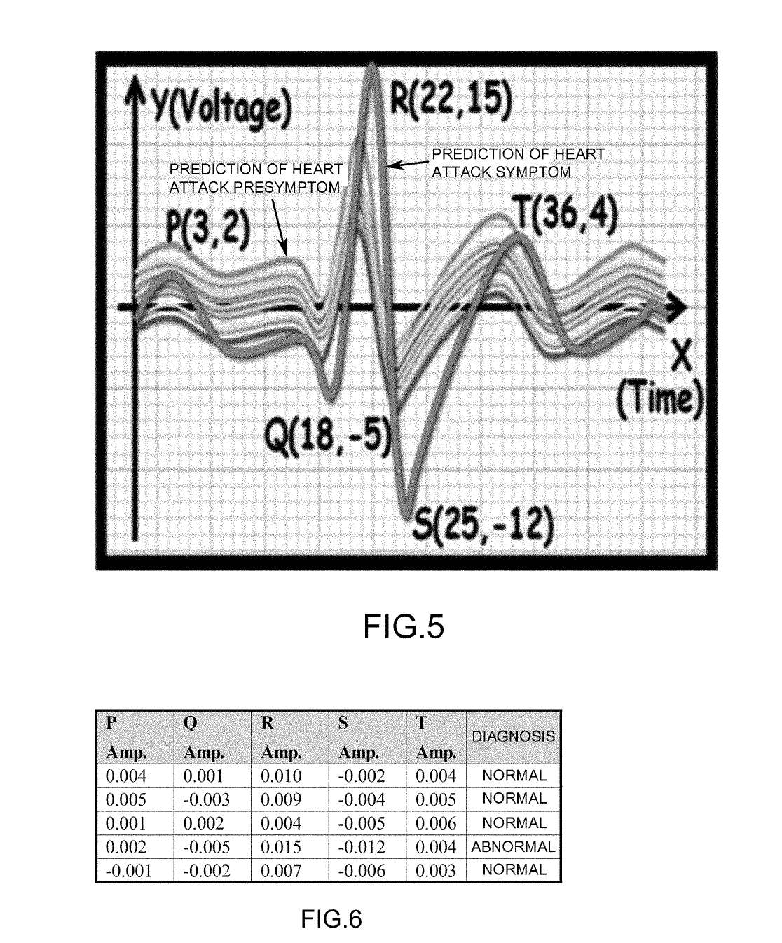

FIG. 5 is an ECG waveform illustrating an ECG pattern representing whether to deviate from a normal condition, and FIG. 6 is a Y-axis voltage table illustrating an abnormality symptom which has deviated from a normal condition according to the present invention.

Referring to FIGS. 5 and 6, the ECG signal processing unit 116 may first amplify an amplitude of a digital electrical signal though signal amplification. The ECG signal processing unit 116 may process the amplified digital electrical signal as a digital electrical signal having a specific waveform. The digital electrical signal may be classified based on a predetermined voltage level, and may be displayed as a continuous ECG pattern having a P waveform, a Q waveform, an R waveform, an S waveform, and a T waveform.

Meanwhile, according to FIG. 6, it may be seen that a value of change is great in the Q, R, and S (QRS) waveforms among the P, Q, R, S, and T waveforms. The present invention may largely use the ECG QRS (X, Y) pattern.

For example, a heart has two atria and two ventricles. The heart generates micro electricity every predetermined period with energy obtained from food, and beats in a sequence of P, QRS, and T waveforms. As such, the heart may have a cardiac cycle which is from a predetermined heartbeat to a next heartbeat, and the cardiac cycle may be divided into atrial systolic, ventricular systolic, and atrial/ventricular diastolic phases. A left atrium and a right atrium may contract and a left ventricle and a right ventricle may relax in the atrial systolic phase, the left atrium and the right atrium may relax and the left ventricle and the right ventricle may contract in the ventricular systolic phase, and all of the left and right atria and the left and right ventricles may relax in the atrial/ventricular diastolic phase.

An ECG may be expressed as a wave frequency in which an activity current and an activity potential difference according to a cardiac contraction are recorded as a wave curve. An ECG waveform may include an upward pulse and a downward pulse which are alternately repeated, and the pulses may be referred to as a P waveform, a Q waveform, an R waveform, an S waveform, and a T waveform described above in order.

Here, the P waveform may be a waveform recording a contraction procedure of the left and right atria, the QRS waveform may be a waveform recording a contraction procedure of the left and right ventricles, and the T waveform may be a waveform recording a relaxation procedure of the left and right ventricles. The P waveform may be generated during depolarization of the atria, the QRS waveforms may be generated during depolarization of ventricles, and the T waveform may be generated during repolarization of the ventricles.

Depolarization of the atria and the ventricles and repolarization of the ventricles of the heart may be measured at the skin of a user. Accordingly, the present invention may measure an effect of the depolarization and the repolarization using the ECG measurement sensor 112 and the smart device 110b.

As an example, the P, Q, R, S, and T waveforms may be extracted at an vertex point at which an upward pulse changes to a downward pulse (or at which the downward pulse is changed to the upward pulse) in the ECG pattern, and may be displayed as an (X, Y) coordinate value by extracting the vertex point (feature point). For example, as shown in FIG. 5, a coordinate value (for example, P(3, 2), Q(18, 5), R(22, 15), S(25, −12), T(22, 15), and T(36, 4)) of each pulse value may be expressed using a grid box.

A health condition of the user may be determined using a coordinate value by extracting a feature point of an ECG pattern extracted from a digital ECG waveform and representing the extracted feature point as the coordinate value, and the coordinate value may be used as information for determining whether the health condition is normal, whether medical care of a doctor is needed, or whether immediate first-aid treatment is needed as an emergency situation by comparing coordinate value with each reference pattern in the abnormality symptom type DB.

The communication unit 118 may have various communication functions such as communication through a mobile network or near field wireless communication, and a real-time heart health learning and predicting management system including a wireless communication module such as a Bluetooth module, an infrared communication module, a ZigBee module, etc. which can perform wireless data communication may store and communicate data in its own wearable smart watch.

A GPS reception unit 120 may receive satellite information including a position of a satellite and time information, and calculate its own position. Since position accuracy is decreased when using only a basic function of receiving and calculating a current position from the satellite, position accuracy may be improved by receiving a correction signal through the communication unit 118.

Although not shown in the drawings, the sudden heart attack prevention system 100 may further include a relay system for relaying a communication of the wearable heart abnormality symptom sensing smart watch 110. The relay system may collect various kinds of information input from the wearable heart abnormality symptom sensing smart watch 110 of a user, communicate with the real-time heart health learning and predicting management system or receive various kinds of health information that periodically provide various notifications at a hospital server, and transmit the information to each wearable heart abnormality symptom sensing smart watch 110.

Further, the relay system may use a cloud server and a DB operating in a cloud computing environment.

Heart health condition information of a user may be provided to an acquaintance 160 or an institution 170. The acquaintance 160 may include a family member, a guardian, or a friend. The institution 170 may include a hospital 170a or a 119 fire station 170b.

Hereinafter, a method of predicting and sensing an emergency condition according to a sudden heart attack using an ECG according to the present invention will be described.

A notification/first-aid treatment method of a heart health condition of a user of the wearable heart abnormality symptom sensing smart watch 110 according to the present invention may be implemented using a sudden heart attack prediction and detection application APP installed in the wearable heart abnormality symptom sensing smart watch 110 described above. The sudden heart attack prediction and detection application APP may be executed based on Android or iPhone.

Accordingly, according to the method of predicting and sensing an emergency condition according to a sudden heart attack using an ECG of the present invention, the method may be autonomously learned by the APP, and may predict and determine an abnormality symptom.

More than 70% of patients may have a presymptom of a heart attack one hour to four weeks before a heart attack occurs even in a sudden heart attack. Usually, presymptoms such as chest pain, dyspnea, nausea and dizziness occur. The presymptom may be expressed by an ECG pattern representing a sudden heart attack preliminary condition.

In this case, heart condition information of a user may be transmitted to the hospital 170a, and a doctor may provide a notification so that the user visits the hospital 170a him/herself if possible or looks for stability by communicating with the user. Further, the heart condition information of the user may be transmitted to a family member, a guardian, or the acquaintance 160.

When a sudden heart attack occurs, cardiopulmonary resuscitation (CPR) should be performed within five minutes. When a heart attack occurs, oxygen supply to the brain is stopped since the heart stops, and first-aid treatment should be performed during the so-called “golden time” since the brain can endure oxygen deprivation for only a few minutes.

Further, the smart device 110b may guide the use of an electric defibrillator installed in the vicinity by informing a person present in the vicinity of the fact (a notification in which a heart attack occurs and first-aid treatment is needed for the user) using a speaker, a light emitting diode (LED), etc.

When a heart attack symptom is present and an ECG pattern is recognized as an emergency, the wearable heart abnormality symptom sensing smart watch 110 may immediately transmit this fact to the 119 fire station 170b. The 119 fire station 170b may detect a position of the wearable heart abnormality symptom sensing smart watch 110 using GPS information and track a current position of the user. The 119 fire station 170b which is closest to the user may immediately go to the user and perform CPR.

As such, since a heart attack presymptom and a heart attack symptom are recognized using the ECG pattern, the sudden heart attack prevention system 100 using an ECG may request cooperation of the acquaintance 160 and the institution 170 so that the user immediately visits the hospital 170a or first-aid treatment is performed even when there is no rescue request of the user.

For example, when the ECG Yellow pattern type representing a heart attack preliminary condition stored in the abnormality symptom type DB is found, the sudden heart attack prevention system 100 using the ECG may recognize that the heart health condition is abnormal and may cause the user to visit the hospital 170a through the user himself/herself or the acquaintance 160 or to take a rest.

When the ECG Red pattern type representing a sudden heart attack generation condition is found, the sudden heart attack prevention system 100 using the ECG may recognize that the heart health condition is an emergency and cause the 119 fire station 170b to immediately perform first-aid treatment for the user.

For example, amplitude of an ECG pattern may be measured. That is, a voltage value generated by depolarization of the heart may be measured, and a corresponding voltage value may be determined as excessively great by being compared to a reference value. An vertex point of an R waveform and an vertex point of an S waveform may be measured among P, Q, R, S, and T waveforms, and a heart health condition may be determined by comparing the measured values and reference values.

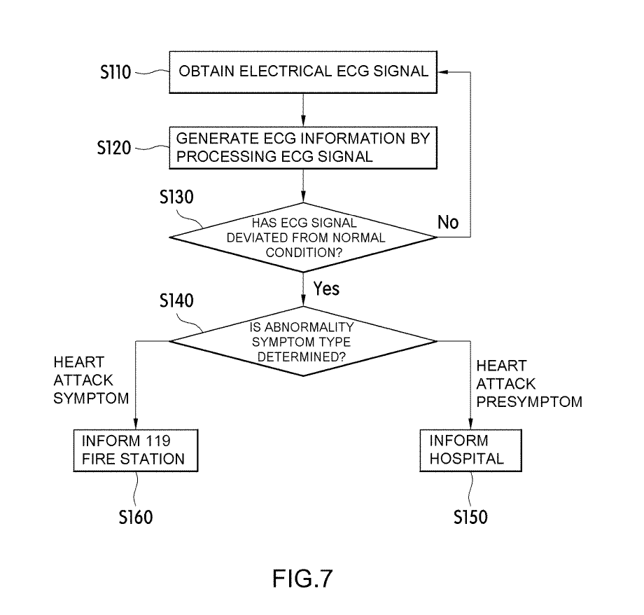

FIG. 7 is a flowchart for describing a method of determining a heart abnormality condition.

Referring to FIG. 7, the ECG signal acquisition unit 114 may collect a digital electrical ECG signal through the ECG measurement sensor 112 (S110). The ECG signal processing unit 116 may process the digital electrical ECG signal, and generate digital ECG information (S120). Next, whether the digital ECG information deviates from a normal condition may be detected (S130). When the digital ECG information deviates from the normal condition, the digital ECG information may be compared with an abnormality symptom type (S140). When the digital ECG information is determined to be a heart attack presymptom, an acquaintance and a hospital may be informed of the digital ECG information (S150). On the other hand, when the digital ECG information is determined to be a heart attack symptom, a 119 fire station may be informed of the digital ECG information (S160).

As described above, the following effects may be obtained according to the configuration of the present invention.

First, since a user always carries the wearable heart abnormal symptom sensing terminal on his or her wrist, a sudden heart attack occurring while sleeping or taking a bath may be prevented. Particularly, since infants cannot express their heart condition in words, the present invention may be more useful for infants.

Second, according to the sudden heart attack prevention system using an ECG, a rescue institution such as a hospital or a 119 fire station may be connected to through a network, a 119 fire station which is closest to a user using GPS may be called when an abnormality symptom occurs, first-aid treatment for a sudden heart attack, which has a very short golden time, may be performed, rescue of a person in the vicinity may be requested using a speaker or a LED, etc. of a corresponding terminal, and a heart electric defibrillator arranged in the vicinity may be suitably used.

As described above, the present invention may perform ECG informatization so as to be able to be compared with a heart attack presymptom or a sudden heart attack symptom stored in an abnormality symptom type DB by processing a digital electrical ECG signal, and a hospital or a 119 fire station may be informed of each symptom when the digital ECG information is included in the abnormality symptom type. Various modifications by those of ordinary skill in the art may be possible without departing from the technical scope of the present invention.

What is claimed is:

1. A sudden heart attack prevention system using digital electrocardiogram (ECG) coordinates comprising:
a system for normally learning using repetitive heart biometric voltage information changed according to a heart health condition of a user and predicting and sensing a sudden heart attack,
wherein the sudden heart attack prevention system uses a plurality of wearable heart abnormal symptom sensing smart watches which are installed on a wrist of the user and provide heart biometric voltage information of the user, and
the wearable heart abnormality sensing smart watch provides biometric voltage (X, Y) pattern information using the heart biometric voltage information, and the biometric voltage (X, Y) pattern information includes ECG PQRST (X, Y) coordinates as a time axis X-coordinate and a heart voltage axis Y-coordinate.

2. The sudden heart attack prevention system using digital ECG coordinates of claim 1, wherein the wearable heart abnormality symptom sensing smart watch comprises:
an ECG measurement sensor configured as one or more biometric digital (X, Y) electrodes;
an ECG signal acquisition unit configured to collect a digital ECG (X, Y) electrical pattern signal from the biometric electrode; and
an ECG signal processing unit configured to process the digital ECG (X, Y) electrical pattern signal and classify the digital ECG (X, Y) electrical pattern signal as an ECG Green pattern type representing a normal condition, an ECG Yellow pattern type representing a heart attack preliminary condition, and an ECG Red pattern type representing a heart attack generation condition, which are comparable with corresponding types stored in an abnormality symptom type database (DB).

3. The sudden heart attack prevention system using digital ECG coordinates of claim 2, wherein the ECG signal acquisition unit collects a digital electrical signal which is continuously changed on a continuous time X-axis from the biometric electrode,
the ECG signal processing unit processes the digital electrical signal as a continuous ECG digital pattern having a P waveform, a Q waveform, a R waveform, a S waveform, and a T waveform by processing the digital electrical signal as an image based on a predetermined voltage Y-axis level, detects a feature point of each of the P waveform, the Q waveform, the R waveform, the S waveform, and the T waveform at an vertex point changing from a high pulse to a low pulse in the continuous ECG pattern, and represents the extracted feature point as a coordinate value.

4. The sudden heart attack prevention system using digital ECG coordinates of claim 3, wherein, when the ECG Yellow pattern type representing the heart attack preliminary condition stored in the abnormality symptom type DB is found in the digital ECG (X, Y) pattern processed by the ECG signal processing unit, the sudden heart attack prevention system informs a hospital or an acquaintance of a sudden heart attack presymptom.

5. The sudden heart attack prevention system using digital ECG coordinates of claim 4, wherein, when the ECG Red pattern type representing the sudden heart attack generation condition stored in the abnormal symptom type DB is found in the digital ECG (X, Y) pattern processed by the ECG signal processing unit, the sudden heart attack prevention system informs a 119 fire station or the acquaintance of a sudden heart attack symptom.

6. A sudden heart attack prevention method using digital electrocardiogram (ECG) coordinates comprising:
obtaining a digital ECG electrical signal;
generating digital ECG (X, Y) pattern information by processing the digital ECG electrical signal;
sensing whether the digital ECG (X, Y) pattern information deviates from a normal condition;
when the digital ECG (X, Y) pattern information deviates from the normal condition, comparing the digital ECG (X, Y) pattern information and an abnormality symptom type; and
when the digital ECG (X, Y) pattern information is determined to be a heart attack presymptom, informing an acquaintance and a hospital of the digital ECG (X, Y) pattern information.

7. The sudden heart attack prevention method using digital ECG coordinates of claim 6, further comprises:
when the digital ECG (X, Y) pattern information is determined to be a heart attack symptom, informing a 119 fire station of the digital ECG (X, Y) pattern information.

8. A wearable heart abnormal symptom sensing smart watch comprising:
a smart band in which an electrocardiogram (ECG) measurement sensor is installed, and which is configured to measure an ECG signal by coming into contact with a wrist of a user; and
a smart device fixed on the wrist using the smart band, configured to process and provide the ECG signal by being in electrical contact with the ECG measurement sensor.

9. The wearable heart abnormal symptom sensing smart watch of claim 8, wherein the ECG signal is a digital electrical signal which is continuously changed on a continuous time X-axis, and the ECG information is a continuous ECG pattern having a P waveform, a Q waveform, a R waveform, a S waveform, and a T waveform which is generated by processing the digital electrical signal as an image based on a predetermined voltage level.

10. The wearable heart abnormal symptom sensing smart watch of claim 9, wherein the ECG information is a feature point of each of the P waveform, the Q waveform, the R waveform, the S waveform, and the T waveform detected at respective vertex points in the ECG pattern, and includes an ECG (X, Y) pattern coordinate value extracted from each of the feature points.
